(19) United States
(12) Patent Application Publication
(10) Pub. No.: US 2009/0325871 A1
(43) Pub. Date: Dec. 31, 2009

(54) Role of the Mk2 Pathway in Wound Repair

(76) Inventor: Usamah Kayyali, Belmont, MA (US)

Correspondence Address: KEOWN & ZUCCHERO, LLP, 500 WEST CUMMINGS PARK, SUITE 1200, WOBURN, MA, 01801, US

(21) Appl. No.: 12/224,865

(22) PCT Filed: Mar. 8, 2007
(86) PCT No.: PCT/US07/06009

§ 371 Date: Apr. 13, 2009

Related U.S. Application Data
(60) Provisional application No. 60/780,302, filed on Mar. 8, 2006.

(51) Int. Cl. A61K 38/16; C07K 14/00; C12Q 1/02; G01N 33/566; A61P 17/02

(52) U.S. Cl. 514/12; 530/350; 435/29; 435/7.1

(57) ABSTRACT

The invention provides a method for screening for a compound that modulates the expression and/or activity of MK2 and the use of the compound for treating a vertebrate that has been wounded.

### RELATED APPLICATIONS

This application is a U.S. National Stage Application submitted under 35 U.S.C. 371 of International Application Serial Number PCT/US2007/006009, filed on Mar. 8, 2007 which claims the benefit of U.S. Provisional Application Ser. No. 60/780,302, filed on Mar. 8, 2006, the contents of which are incorporated herein by reference in its entirety.

### GOVERNMENT FUNDING

This invention was made with government support under HL-79320 awarded by the National Institutes of Health. The government has certain rights in the invention.

### BACKGROUND OF THE INVENTION

### Summary of the Related Art

Fibroblasts play a major role in normal physiology and disease processes such as wound repair, scarring, and fibrosis.

Differentiation of fibroblasts into myofibroblasts has been a subject of interest because of the role this step is believed to play in wound healing. Several signaling pathways have been implicated in this process. Myofibroblasts are identified by increased expression of smooth muscle α-actin (αSMA or smα), which is usually expressed in smooth muscle cells. Functionally, myofibroblasts are more contractile and structurally they are characterized by increased actin stress fibers both of which are believed to be due to the increased expression of smα (for review, see Hinz, B., and Gabbiani, G. Mechanisms of force generation and transmission by myofibroblasts. Curr Opin Biotechnol 14, 538-546 (2003)). Thus the mechanism by which smα expression is regulated has received considerable attention.

Myofibroblasts derive from fibroblasts through the action of growth factors, such as, TGFβ (Desmouliere, A., et. al., Transforming growth factor-beta 1 induces alpha-smooth muscle actin expression in granulation tissue myofibroblasts and in quiescent and growing cultured fibroblasts. J Cell Biol 122, 103-111 (1993); Ronnov-Jessen, L., and Petersen, O. W. Induction of alpha-smooth muscle actin by transforming growth factor-beta 1 in quiescent human breast gland fibroblasts. Implications for myofibroblast generation in breast neoplasia. Lab Invest 68, 696-707 (1993); Roy, S. G. et. al., Regulation of [alpha]-smooth muscle actin gene expression in myofibroblast differentiation from rat lung fibroblasts. The International Journal of Biochemistry & Cell Biology 33, 723 (2001); Yokozeki, M. et. al., Transforming growth factor-beta 1 modulates myofibroblastic phenotype of rat palatal fibroblasts in vitro. Exp Cell Res 231, 328-336 (1997)). The regulation of smα expression in fibroblasts and other cell types is quite complex and at the transcriptional level alone involves several interacting factors, enhancers and suppressors. The promoter region of the smα gene contains elements (SRE) that are responsive to serum response factor (SRF). The CArG box, which is a core component of the SRE has been shown to be responsive to serum in the mouse (Foster, D. N., et al., Positive and negative cis-acting regulatory elements mediate expression of the mouse vascular smooth muscle alpha-actin gene. The Journal Of Biological Chemistry 267, 11995 (1992); Stoflet, E. S. et. al., Activation of a muscle-specific actin gene promoter in serum-stimulated fibroblasts. Mol Biol Cell 3, 1073-1083 (1992)), as well as, the human (Kim, J. H., et. al., Smooth Muscle [alpha]-Actin Promoter Activity Is Induced by Serum Stimulation of Fibroblast Cells. Biochemical and Biophysical Research Communications 190, 1115 (1993)) smα promoter in fibroblasts. However, while mutation of the CArG sites in the promoter abolished its activity in smooth muscle cells in vitro and in vivo (Mack, C. P., and Owens, G. K. Regulation of Smooth Muscle {alpha}-Actin Expression In Vivo Is Dependent on CArG Elements Within the 5′ and First Intron Promoter Regions. Circ Res 84, 852-861 (1999)), it had no effect in endothelial cells suggesting that regulation varies in different cell types (Shimizu, R. T. et. al., The Smooth Muscle alpha-Actin Gene Promoter Is Differentially Regulated in Smooth Muscle versus Non-smooth Muscle Cells. J Biol Chem 270, 7631-7643 (1995)). In addition to the CArG, a TGFβ responsive element (TCE) has been reported that enhanced SRF binding and appeared to be necessary for TGFβ induction of smα in smooth muscle cells (Hautmann, M. B., et. al., A transforming growth factors (TGFβ) control element drives TGFβ-induced stimulation of smooth muscle α-actin gene expression in concert with two CArG elements. Journal of Biological Chemistry 272, 10948 (1997)) and fibroblasts (Hautmann, M. B., et. al., Similarities and differences in smooth muscle [alpha]-actin induction by TGF-[beta] in smooth muscle versus non-smooth muscle cells. Arteriosclerosis, Thrombosis, and Vascular Biology 19, 2049 (1999)). One study also suggested that CArG is less important for induction of smα in fibroblasts treated with TGFβ than TCE (Roy, S. G. et. al., Regulation of [alpha]-smooth muscle actin gene expression in myofibroblast differentiation from rat lung fibroblasts. The International Journal of Biochemistry & Cell Biology 33, 723 (2001)). Recent studies suggested that the TCE site in the smα promoter mediates both activation and repression of the gene (Liu, Y., et. al., A Transforming Growth Factor-{beta} Control Element Required for SM {alpha}-Actin Expression in Vivo Also Partially Mediates GKLF-dependent Transcriptional Repression. J Biol Chem 278, 48004-48011 (2003)). In addition, Smad3 which mediates some of TGFβ effects has been reported to bind to Smad3-binding elements in the smα promoter and activate it (Hu, B., et. al., Smad3 Mediates Transforming Growth Factor-{beta}-Induced {alpha}-Smooth Muscle Actin Expression. Am J Respir Cell Mol Biol 29, 397-404 (2003)). Several other studies suggested that repressor proteins such as, the Pur family (Subramanian, S. V. et. al., Induction of Vascular Smooth Muscle {alpha}-Actin Gene Transcription in Transforming Growth Factor {beta} 1-Activated Myofibroblasts Mediated by Dynamic Interplay between the Pur Repressor Proteins and Sp1/Smad Coactivators. Mol Biol Cell 15, 4532-4543 (2004)), MYSI (Kelm, R. J., Jr., et. al., Molecular Interactions between Single-stranded DNA-binding Proteins Associated with an Essential MCAT Element in the Mouse Smooth Muscle alpha-Actin Promoter. J Biol Chem 274, 14238-14245 (1999); Kelm, R. J., Jr., et. al., Structure/Function Analysis of Mouse Pur{beta}, a Single-stranded DNA-binding Repressor of Vascular Smooth Muscle {alpha}-Actin Gene Transcription. J Biol Chem 278, 38749-38757 (2003)) and its human analog YB-1 (Zhang, A. et. al., YB-1 Coordinates Vascular Smooth Muscle {alpha}-Actin Gene Activation by Transforming Growth Factor {beta} 1 and Thrombin during Differentiation of Human Pulmonary Myofibroblasts. Mol Biol Cell 16, 4931-4940 (2005)) also contributed to the complex tissue specific expression of smα possibly through interacting with SRF, SP I and Smad2/3. Since TGFβ has been described to activate p38 in fibroblasts and since SRF activation has been shown to be p38 dependent (Deaton, R. A., et. al., Transforming Growth Factor-{beta} 1-induced Expression of Smooth Muscle Marker Genes Involves Activation of PKN and p38 MAPK. J Biol Chem 280, 31172-31181 (2005)), activation of SRF was focused on as a mechanism that might explain the different levels of smα expression in wild type versus MK2^{-/-} fibroblasts. Indeed one report demonstrating that MK2 directly phosphorylates and activates SRF (Heidenreich, O., et. al., MAPKAP Kinase 2 Phosphorylates Serum Response Factor in Vitro and in Vivo. J Biol Chem 274, 14434-14443 (1999)) suggested a direct link between p38 activation and sma expression. Thus, it is hypothesized that the reason MK2^{-/-} fibroblasts lack smα is due to their inability to activate SRE in the promoter region of the smα gene since SRF in these cells is not being phosphorylated. Differences in the phosphorylation level of SRF between MK2^{-/-} versus WT fibroblasts could not be identified either under normal conditions or in response to TGFβ (data not shown). Still, whether MK2 level might affect the SRE indirectly by expressing SRE-luciferase in WT and TGFβ fibroblasts was tested.

Both WT and MK2^{-/-} fibroblasts expressed significant activity of the SRE promoter when stable transfectants were generated (Sousa, A. M. et. al., Smooth muscle alpha-actin expression and myofibroblast differentiation by TGFbeta are dependent upon MK2. J Cell Biochem (2006)). Moreover, a significant difference in the extent or time-course of activation of the smα promoter by TGFβ in either cell type was not observed (Sousa, A. M. et. al., Smooth muscle alpha-actin expression and myofibroblast differentiation by TGFbeta are dependent upon MK2. J Cell Biochem (2006)). These observations lead to conclusion that MK2 affects smα by mechanisms that are independent of the SRE or SRF. The fact that a p38 inhibitor was able to block SRE promoter activation (more so in the MK2^{-/-} MEF than in WT) led to the postulate that the p38-mediated activation of SRF/SRE by TGFβ remained functional after disrupting MK2 expression. Hence, MK2 might affect smα expression through actions on other transcription enhancers or repressors.

MK2 might regulate smα expression by posttranscriptional mechanisms. For example MK2 has been shown to increase IL-6 and TNF message stability, as well as TNF translation upon induction by lipopolysaccharide (Hitti, E., et. al., Mitogen-activated protein kinase-activated protein kinase 2 regulates tumor necrosis factor mRNA stability and translation mainly by altering tristetraprolin expression, stability, and binding to adenine/uridine-rich element. Mol Cell Biol 26, 2399-2407 (2006); Kotlyarov, A., and Gaestel, M. Is MK2 (mitogen-activated protein kinase-activated protein kinase 2) the key for understanding post-transcriptional regulation of gene expression? Biochem Soc Trans 30, 959-963 (2002); Neininger, A., et. al., MK2 Targets AU-rich Elements and Regulates Biosynthesis of Tumor Necrosis Factor and Interleukin-6 Independently at Different Post-transcriptional Levels. J Biol Chem 277, 3065-3068 (2002)).

As described above, one of the characteristics of fibroblasts is their ability to differentiate into myofibroblasts, a contractile form that is identified by increased production of smooth muscle α-actin (smα). Regardless of the exact mechanism by which smα expression is regulated in fibroblasts interfering with that mechanism is likely to perturb the balance between fibroblasts versus myofibroblasts. As that balance is critical for wound healing, modulating smα expression is likely to be a viable approach in managing the latter conditions. There is, therefore, a need for the ability to restore and/or increase MK2 activity and modulate smα expression.

### BRIEF SUMMARY OF THE PREFERRED EMBODIMENTS

In a first aspect the invention provides a method for therapeutically treating a vertebrate that has been wounded. The method in this aspect of the invention comprises administering to the vertebrate a compound that increases the expression and/or activity of MK2, thereby aiding in the wound healing or wound repair processes. In certain embodiments of the invention the vertebrate is a mammal, preferably a human.

In a second aspect the invention provides a method for screening for a compound that modulates the expression and/or activity of MK2. The method according to this aspect of the invention comprises providing cells, contacting the cells with a test compound and assaying the cells for changes in expression and/or activity of MK2 in the presence of the compound. As used herein, the term “modulates” means to change or alter the activity and/or expression of MK2 in the presence of the compound as compared to the absence of the compound. The alteration or change can either be an increase (i.e., stimulation) or decrease (i.e., inhibition) of the expression and/or activity of MK2 in the presence of the compound. Targeting MK2 with a compound that increases the expression and/or activity of MK2 can present a therapeutic approach to improving myofibroblasts role in conditions where such increase is beneficial, such as wound repair Whereas, targeting MK2 with a compound that inhibits the expression and/or activity of MK2 can present a therapeutic approach to managing conditions where MK2 activity is harmful.

In a third aspect the invention provides for compounds identified by the screen in the second aspect of the invention either alone or as part of a pharmaceutical composition.

### BRIEF DESCRIPTION OF THE DRAWINGS

FIG. 1: MK2−/− MEF do not express the MK2 protein. Cell lysates from WT or MK2−/− MEF were immunoblotted with an anti-MK2 antibody. While significant bands were observed in WT MEF, MK2 protein was not detectable in the MK2−/− cells, either at baseline or after treatment with TGFβ.

FIG. 2: Disruption of MK2 expression reduces actin stress fibers. Wild type and MK2−/− fibroblasts were grown on glass coverslips and then stained with rhodamine-phalloidin. MK2−/− MEF contained less filamentous actin than wild type MEF in which actin was more organized into parallel stress fibers. Figure shows a representative area of several coverslips from several experiments.

FIG. 3: Most MK2−/− fibroblasts do not express alpha-smooth muscle actin (sma). Wild type and MK2−/− fibroblasts were grown on glass coverslips and then stained with rhodamine-phalloidin (red) and anti smα antibody (green). Most wild type fibroblasts contained smα which colocalized with filamentous actin (yellow), while the majority of MK2 fibroblasts did not contain any sma.

FIG. 4: MK2−/− MEF contain less smα-actin than Wild Type MEF. The level of pan-actin which is not smooth muscle- or myofibroblast-specific did not appear to differ between the two cell types.

FIG. 5: TGFβ treatment increases smα-actin production in WT MEF but not in MK2−/− MEF. When WT MEF were treated with TGFβ (1 ng/mL for 24 hours), smα-actin level increased consistent with the myofibroblast differentiating effect of TGFβ on fibroblasts. MK2−/− MEF on the other hand, did not respond to TGFβ by increasing smα-actin, but rather appeared to express less smα-actin in TGFβ-treated MK2−/− MEF than in vehicle-treated cells. The MK2−/− blot was overexposed to reveal the difference due to TGFβ treatment and does not reflect the difference between wild type and MK2−/− smα-actin levels described earlier.

FIG. 6: TGFβ produces opposite effects in MK2−/− MEF versus WT fibroblasts. The level of smα and β-actin mRNA were determined by RT-PCR on equal amounts of RNA isolated from WT or MK2−/− MEF treated with or without TGFβ (1 ng/mL) for 24 hours. MK2−/− MEF contained less smα mRNA than WT MEF at baseline. TGFβ increased smα mRNA in WT MEF while it significantly reduced it in MK2−/− MEF.

FIG. 7: Collagen message levels are not affected by MK2 suppression. The level of procollagen type I α1 mRNA were determined by RT-PCR on equal amounts of RNA isolated from WT or MK2−/− MEF treated with or without TGFβ (1 ng/mL) for 24 hours. MK2−/− MEF contained comparable levels of message as WT MEF at baseline. TGFβ did not appear to alter collagen mRNA significantly in either cell types after 24 hours of exposure.

FIG. 8: TGFβ treatment increases SRE-luciferase promoter activity in WT MEF. When WT MEF were treated with TGFβ (1 ng/mL for shown duration), SRE promoter activity increased with the greatest activation observed after 6 hours of treatment (A). SB203580, a p38 inhibitor, partially blocked TGFβ activation of SRE promoter measured at 6 hours. * indicates statistically significant difference from Control mean, # indicates statistically significant difference from TGFβ+SB mean; a<0.05.

FIG. 9: TGFβ treatment increases SRE-luciferase promoter activity in MK2−/− MEF. When MK2−/− MEF were treated with TGFβ (1 ng/mL for shown duration), SRE promoter activity increased similarly to WT MEF with the greatest activation observed after 6 hours of treatment (A). SB203580, a p38 inhibitor, completely blocked TGFβ activation of SRE promoter measured at 6 hours. * indicates statistically significant difference from Control mean, # indicates statistically significant difference from TGFβ+SB mean; a<0.05.

FIG. 10: smα mRNA is less stable in MK2^{-/-} MEF than in WT MEF. MEF were incubated with actinomycin D (2.4 μM) for 0, 2, 4, 6, 8, 16, and 24 hours; then smα mRNA levels relative to β-actin mRNA were determined. Single-phase exponential decay regression lines are derived from 3 samples for each exposure group. The solid line and open circles represent WT MEF, and the dashed line and closed diamonds represent MK2^{-/-} MEF. Regression curves were obtained from the exponential decay of mRNA using the formula y=ae^{-bx} where a is the smα mRNA value at time 0 (100%) and b the decay rate constant for each condition. smα mRNA half-life for each curve was calculated as t_{1/2}=_[lna−ln50]/b.

FIG. 11: MK2^{-/-} MEF proliferate faster than wild type MEF. When wild type and MK2^{-/-} MEF were seeded at equal density, and counted after 24, 48 and 72 hours, MK2^{-/-} MEF cell count increased faster than wild type MEF cell count.

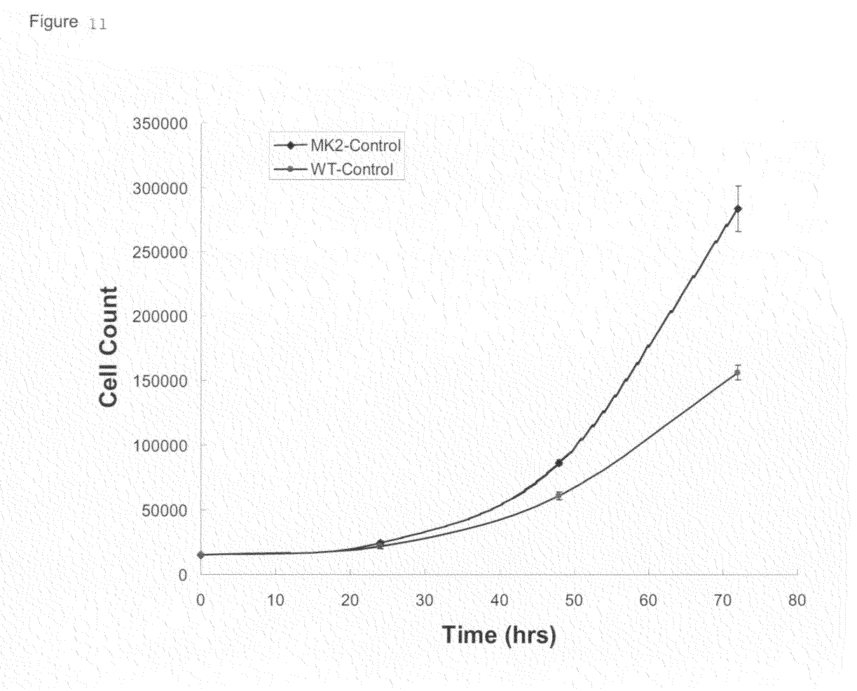

FIG. 12: MK2^{-/-} MEF are slower than wild type MEF in repopulating a wounded part of the culture dish. Cells were scraped off sub-confluent MEF culture dishes of equal density using a sterile pipette tip. After 24 hours more wild type MEF than MK2^{-/-} MEF were observed in the wounded area. Over expressing phosphomimicking pmHSP27 in MK2−/− cells did not reverse the defective migratory properties of MK2^{-/-} MEF.

### DETAILED DESCRIPTION OF THE PREFERRED EMBODIMENTS

The invention relates to therapeutic approaches to managing conditions related to MK2 activity. The patents and publications cited herein reflect the level of knowledge in the art and are hereby incorporated by reference in their entirety. Any conflict between the teachings of these patents and publications and this specification shall be resolved in favor of the latter.

Without wishing to be bound to any particular theory, the results presented below indicate that MK2 plays an important role in myofibroblast differentiation as defined by the expression of smα. In particular it has been demonstrated that embryonic fibroblasts derived from MK2-deficient mice lack stress fibers and smα. In addition, unlike their wild type counterparts the cells do not respond to TGFβ by increasing smα production but by suppressing it. These findings might implicate MK2 in wound healing.

In a first aspect the invention provides a method for therapeutically treating a vertebrate that has been wounded. The method in this aspect of the invention comprises administering to the vertebrate a compound, either alone or as part of a pharmaceutical composition, that increases the expression and/or activity of MK2, thereby aiding in the wound healing or wound repair processes. In certain embodiments of the invention the vertebrate is a mammal, preferably a human.

As used herein, the term “wounded” is type of physical trauma or injury wherein the skin is torn, cut or punctured (an open wound), or where blunt force trauma causes a contusion (a closed wound).

As used herein, the terms “wound healing” or “wound repair” refers to the body's natural process of regenerating dermal and epidermal tissue. Without wishing to be bound to any particular theory, when an individual is wounded, a set of events takes place in a predictable fashion to repair the damage. These events overlap in time and have been artificially categorized into separate steps: the inflammatory, proliferative, and maturation phases. In the inflammatory phase, bacteria and debris are phagocytized and removed and factors are released that cause the migration and division of cells involved in the proliferative phase. The proliferative phase is characterized by angiogenesis, collagen deposition, granulation tissue formation, epithelialization, and wound contraction. In angiogenesis, new blood vessels grow from endothelial cells. In fibroplasia and granulation tissue formation, fibroblasts grow and form a new, provisional extracellular matrix (ECM) by excreting collagen and fibronectin. In epithelialization, epithelial cells crawl across the wound bed to cover it. In contraction, the wound is made smaller by the action of myofibroblasts, which establish a grip on the wound edges and contract themselves using a mechanism similar to that in smooth muscle cells. When the cells' roles are close to complete, unneeded cells undergo apoptosis. In the maturation and remodeling phase, collagen is remodeled and realigned along tension lines and cells that are no longer needed are removed by apoptosis.

In the methods according to this invention, administration of the compound can be by any suitable route, including, without limitation, parenteral, oral, sublingual, transdermal, topical, inhalation, intranasal, aerosol, intraocular, intratracheal, intrarectal or vaginal. Administration of the therapeutic compositions the compound of the invention can be carried out using known procedures at dosages and for periods of time effective to treat the wound or promote healing of the wound. The term an “effective amount” or a “sufficient amount” generally refers to an amount sufficient to affect a desired biological effect, such as beneficial results. Thus, an “effective amount” or “sufficient amount” will depend upon the context in which it is being administered. It may be desirable to administer simultaneously, or sequentially a therapeutically effective amount of one or more of the therapeutic compositions of the invention to an individual as a single treatment episode.

In the methods according to this invention, the compound can be administered in combination with any other agent useful for treating a wound or promoting wound repair that does not diminish the effect of the compound. Agents known in the art to be useful for treating a wound or facilitating wound repair include, but are not limited to, antibiotics such as, but not limited to, Bacitracin, tetracycline, gramicidin, gentamicin, and tobramycin; antifungal compounds such as, but not limited to, Amphotericin B and fluconazole; and steriods such as, but not limited to, cortisone.

The term “in combination with” generally means in the course of treating the same condition in the same patient, and includes administering a compound and/or an agent in any order, including simultaneous administration, as well as temporally spaced order of up to several days apart. Such combination treatment may also include more than a single administration of the compound according to the invention, and/or independently an agent. The administration of the compound and/or agent may be by the same or different routes.

In a second aspect the invention provides a method for screening for a compound that modulates the expression and/or activity of MK2. The method according to this aspect of the invention comprises providing cells, contacting the cells with a test compound and assaying the cells for changes in expression and/or activity of MK2 in the presence of the compound as compared to the absence of the compound. As used herein, the term “modulates” means to change or alter the activity and/or expression of MK2 in the presence of the compound as compared to the absence of the compound. The alteration or change can either be an increase (i.e., stimulation) or decrease (i.e., inhibition) of the expression and/or activity of MK2 in the presence of the compound. Targeting MK2 with a compound that increases the expression and/or activity of MK2 can present a therapeutic approach to improving myofibroblasts role in conditions where such increase is beneficial, such as wound repair. Whereas, targeting MK2 with a compound that inhibits the expression and/or activity of MK2 can present a therapeutic approach to managing conditions where MK2 activity is harmful.

Without wishing to be bound by any particular theory, a compound that modulates the expression and/or activity of MK2 and is useful for treating a wound or promoting wound repair can include, but is not limited to, a compound that binds the autoinhibitory peptide of MK2 preventing it from binding MK2 and thus resulting in MK2 activation, or a compound that mimics the MK2 inhibitory peptide for inhibiting MK2. In addition the compound could include those that activate p38 MAP kinase, the upstream activator of MK2, such as hyperosmolar solutions, low oxygen, and sodium arsenite which are expected to cause activation of MK2, as well as a compound that inhibits p38, such as SB203580, which is expected to inhibit MK2. Also, the compound can include a nucleotide sequence that encodes MK2 or a promoter of MK2 as part of a DNA vector or virus that can be used in gene therapy.

In a third aspect the invention provides for compounds identified by the screen in the second aspect of the invention either alone or as part of a pharmaceutical composition. These compositions comprise a compounds identified by the second aspect and a pharmaceutically acceptable carrier.

As used herein, the term “pharmaceutically acceptable” refers to a material that does not interfere with the effectiveness of the compositions of the first or second aspects of the invention and is compatible with a biological system such as a cell, cell culture, tissue, or organism. In certain embodiments, the biological system is a living organism, such as a vertebrate.

As used herein, the term “carrier” encompasses any excipient, diluent, filler, salt, buffer, stabilizer, solubilizer, lipid, or other material well known in the art for use in pharmaceutical formulations. It will be understood that the characteristics of the carrier, excipient, or diluent will depend on the route of administration for a particular application. The preparation of pharmaceutically acceptable formulations containing these materials is described in, e.g., Remington's Pharmaceutical Sciences, 18th Edition, ed. A. Gennaro, Mack Publishing Co., Easton, Pa., 1990, ISBN: 0-912734-04-3.

The examples below are intended to further illustrate certain preferred embodiments of the invention, and are not intended to limit the scope of the invention.

Data are presented as means±standard deviation (SD); n represents the number of cells. Statistical differences were determined by either Student's t-test for comparison of two sample means or analysis of variance (ANOVA) for comparison of more than two sample means followed by Bonferroni post-hoc testing for multiple comparisons between two sample means (P<0.05 was considered statistically significant).

### Example 1

### Materials and Reagents

Dulbecco's modified Eagles's medium (DMEM), fetal bovine serum (FBS), dialyzed FBS, penicillin G potassium, streptomycin, fungizone, and glutamine were purchased from Invitrogen (Carlsbad, Calif.). All other reagents and drugs were obtained from Sigma (St. Louis, Mo.), with the exception of SB-203580-HCl (p38 MAPK inhibitor) and which was purchased from Calbiochem (La Jolla, Calif.). TGFβ was purchased from R&D Systems (Minneapolis, Minn.) and activated before use according to manufacturers instructions. On the day of the experiments, all drugs were diluted in serum-free medium.

### Example 2

### Cell Culture and Transfection

Immortalized mouse embryonic fibroblasts from wild type and MK2^{-/-} knockout mice were prepared as described earlier (Kotlyarov, A., et. al., MAPKAP kinase 2 is essential for LPS-induced TNF-alpha biosynthesis. Nat Cell Biol 1, 94-97 (1999)). In brief, primary embryonic fibroblasts from C57BL/6J wild type and MK2^{-/-} mice, were co-transfected with pSV40Tag encoding the SV40 large T antigen and the pREP8 plasmid, and then, histidinol-resistant colonies were selected and expanded. The cells were maintained in DMEM containing 10% FBS, penicillin, streptomycin, fingizone and glutamine at 37° C. in humidified air containing 5% CO₂. Fibroblasts were passaged in 0.25% trypsin-0.02% ethylenediaminetetraacetic acid (EDTA) solution, and one day prior to the experiments, cells were maintained in serum-free media.

For SRE promoter-reporter assays WT and MK2^{-/-} MEF, were transfected with the SRE.L-luciferase vector which contains two copies of SRE.L, a derivative of c-fos serum response element driving the expression of luciferase. The SRE.L vector was introduced alone in the case of WT MEF, and co-transfected with pMEpuro (a selection vector which confers resistance to puromycin to eukaryotic cells) in the case of MK2^{-/-} MEF. The vectors were introduced (5 μg of SRE.L, and 0.5 μg pMEpuro) into MEF using lipofectamine. Stable transfected WT MEF cell lines were obtained by selection with geneticin, and resistant colonies were isolated, expanded, and then screened for baseline luciferase activity. Since the MK2^{-/-} MEF were already resistant to geneticin, stable transfectants produced by co-transfection with pMEpuro were selected with puromycin, and resistant clones were isolated and expanded as above. The firefly luciferase activity was assayed using a kit from Promega (Madison, Wis.) according to the manufacturer's instructions. In brief, cells were lysed and the substrate (beetle luciferin) was added to the lysate. Next, chemiluminescence was measured using a luminometer.

### Example 3

### MK2 Kinase Assay

After treatment of cells, activation of MK2 was assayed by measuring the activity of the immunoprecipitated enzyme. Specific MK2 activity was assayed using the MK2 assay kit from Upstate Biotechnology (Lake Placid, N.Y.). In brief, the cells were washed and lysed, and then MK2 was immunocomplexed with agarose-conjugated anti-MK2 antibody by rocking overnight at 4° C. The immunocomplex was then brought down by centrifugation and the pellet was washed. Next, MK2-specific peptide substrate was added along with [γ-³²P]ATP and incubated with the immunoprecipitated kinase with vigorous shaking at 30° C. Then, the complex was brought down by centrifugation and the supernatant, which contains the peptide substrate, was spotted on p81 phosphocellulose paper, and washed with phosphoric acid and acetone to remove unincorporated label. Finally, the p81 paper was transferred to scintillation vials containing scintillation cocktail, and the samples were counted on a Packard beta counter. (Ref: Kayyali, U., et. al. Cytoskeletal Changes in Hypoxic Pulmonary Endothelial Cells are Dependent on MAPK-activated Protein Kinase MK2. J. Biol. Chem. 277, 42596-42602, (2002)).

### Example 4

### SDS-PAGE and Western Blotting

Aliquots from the cell lysates prepared as described above were assayed for protein using the Bradford protein assay and then diluted with 2× Laemmli loading buffer for SDS-PAGE. Equal amounts of protein were then loaded in each well of 4-20% Tris/glycine gels. After electrophoresis for 90 min at 125 V constant voltage, the gel was blotted onto an Immobilon-P membrane by electrophoretic transfer at 25 V constant voltage overnight. The membrane was then washed, blocked with 5% milk, and probed with antibodies against MK2 (Cell Signaling, Beverly, Mass.) smα (Sigma, St. Louis, Mo.) or pan-actin (Santa Cruz, Santa Cruz, Calif.). The immunoreactive bands were visualized using a secondary antibody conjugated to horseradish peroxidase (Pierce, Rockford, Ill.).and a chemiluminescent substrate according to the manufacturer's instructions (SuperSignal, Pierce, Rockford, Ill.). The intensity of the bands was quantified using a UnScanIt software (Orem, Utah).

### Example 5

### Quantitation of smα, β-Actin and Procollagen RNA

Semiquantitative RT-PCR to measure mRNA in mouse MEF was used. In brief, RNA was isolated from treated or untreated MEF using the RNeasy kit (Qiagen, Valencia, Calif.) according to manufacturesr's instructions. The amount of RNA was determined spectrophotometrically, and equal amounts were used to generate cDNA by reverse transcription using the Superscript III system (Invitrogen, Carlsbad, Calif.) according to manufacturer's instructions. Next an equal volume of the cDNA was added to a master mix containing the following primers. For sma, the primers 5′-GCATCCACGAAACCACCTA-3′ and 5′-CACGAGTAACAAATCAAAGC-3′ were used to generate a 418 bp PCR product from mouse smooth muscle α-actin cDNA. For β-actin, the primers 5′-TGGAATCCTGTGGCATCCATGAAAC-3′ and 5′-TAAAACGCAGCTCAGTAACAGTCCG-3′) were used to generate 348 bp product from mouseβ-actin cDNA. The primers 5′-GAGCGGAGAGTACTGGATCG-3′ and 5′-GTTCGGGCTGATGTACCAGT3′ were used to generate a 142 bp product from mouse procollagen type I alpha 1 cDNA. The PCR reaction was carried out as using a Bio-Rad I-cycler thermal cycler (Hercules, Calif.). The PCR products were then resolved on a 2% NuSieve 1% SeaKem agarose gel. After staining with ethidium bromide, photographs of the gels were taken, scanned, and the intensity of the bands was measured using UnScanIt software (Orem, Utah). The relative amount of smα and procollagen RNA was determined by dividing the intensity of their corresponding PCR product band by the intensity of the β-actin PCR product band.

### Example 6

### MK2^{-/-} MEF Contain Less Actin Stress Fibers than WT MEF

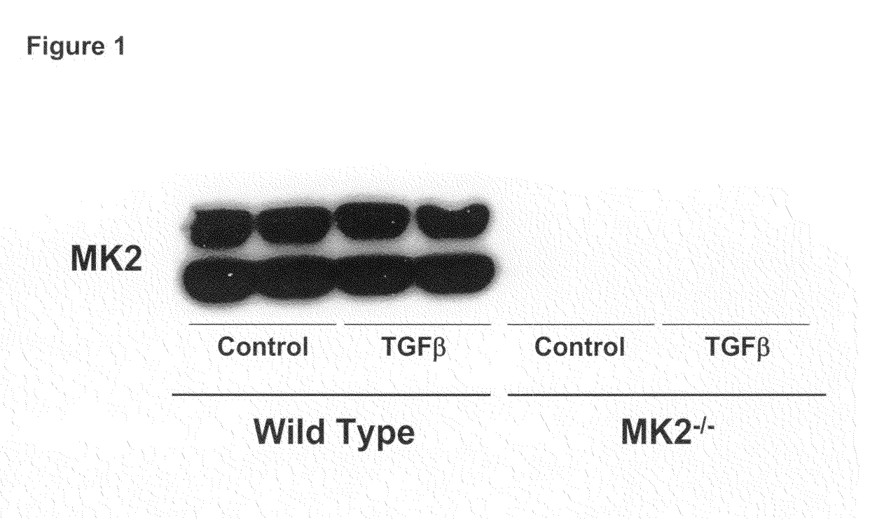

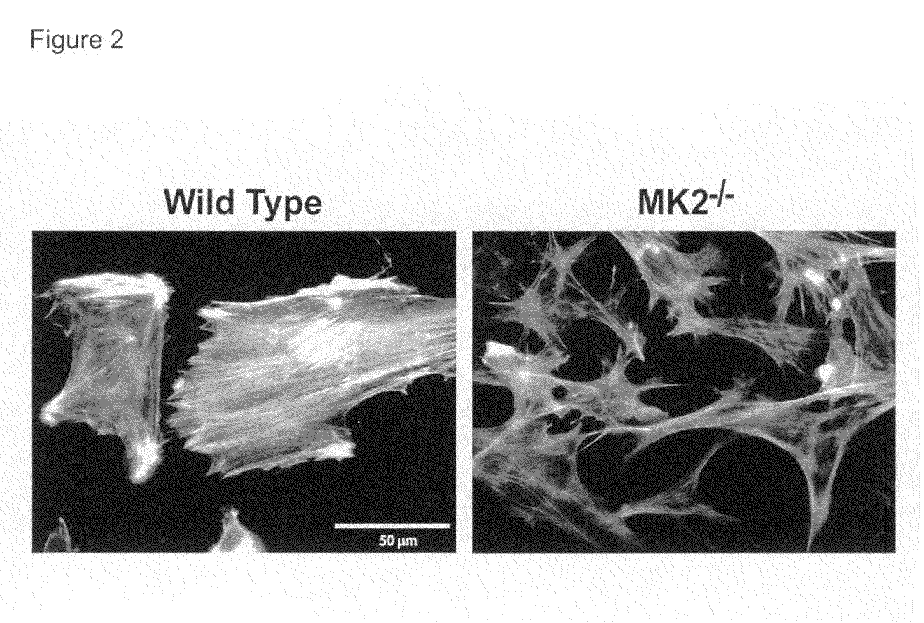

After verifying that MK2 is not expressed in MK2^{-/-} MEF (FIG. 1), MEF were seeded on sterile glass coverslips. At various degrees of confluence, cells were rinsed twice with phosphate-buffered saline (PBS), and fixed for 10 minutes with 4% formaldehyde. Next the cover slips were washed twice with PBS, then permeabilized for 10 minutes with 0.4% triton-X-100 in PBS. The cells were stained with rhodamine-phalloidin (Molecular Probes/Invitrogen, Carlsbad, Calif.) for 20 minutes. The cover slips were then washed with PBS, mounted with Citifluor and examined using a Zeiss fluorescence microscope. As shown in FIG. 2, MEF from wild type mice contain a significant amount of parallel actin stress fibers. MEF from MK2^{-/-} mice, on the other hand, contain significantly less stress fibers and filamentous actin is organized in web like form rather than in parallel fibers.

### Example 7

### Disruption of MK2 Expression Reduces smα Level in Embryonic Fibroblasts

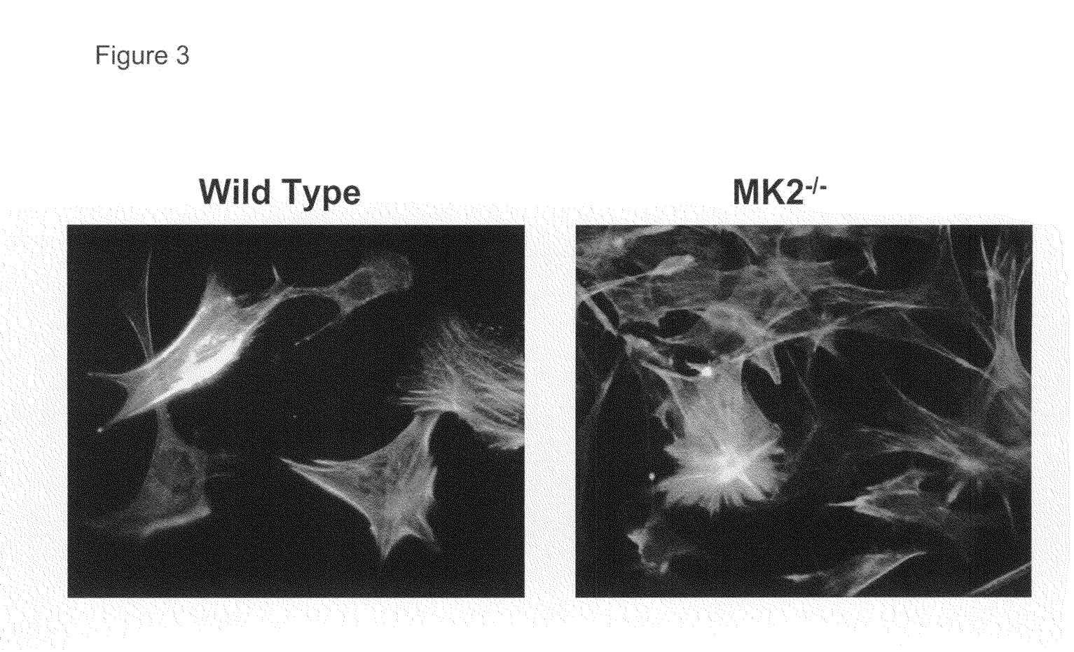

Since increased actin stress fiber formation has been associated with differentiation of fibroblasts into myofibroblasts, whether MK2^{-/-} MEF differed from wild type MEF in other fibroblast/myofibroblast characteristics was examined. One hallmark of differentiation of fibroblasts into myofibroblasts is the increased expression of smα. For immunocytochemistry, after rhodamine phalloidin staining the coverslips were washed with PBS then incubated overnight with mouse anti smα antibody (Calbiochem, La Jolla, Calif.). Next day, the primary antibody was washed and the cells were incubated for 30 min with Alexafluor 582-conjugated anti mouse antibody (Molecular Probes/Invitrogen, Carlsbad, Calif.), washed, mounted with citifluor and examined. As shown in FIG. 3 only few MK2^{-/-} MEF were immunolabeled with anti-smα antibody while the majority of WT MEF stained positive for smα. Furthermore, smα colocalized with rhodamine-phalloidin stained stress fibers in WT MEF, while it did not colocalize with filamentous actin in the few smα-positive MK2 MEF.

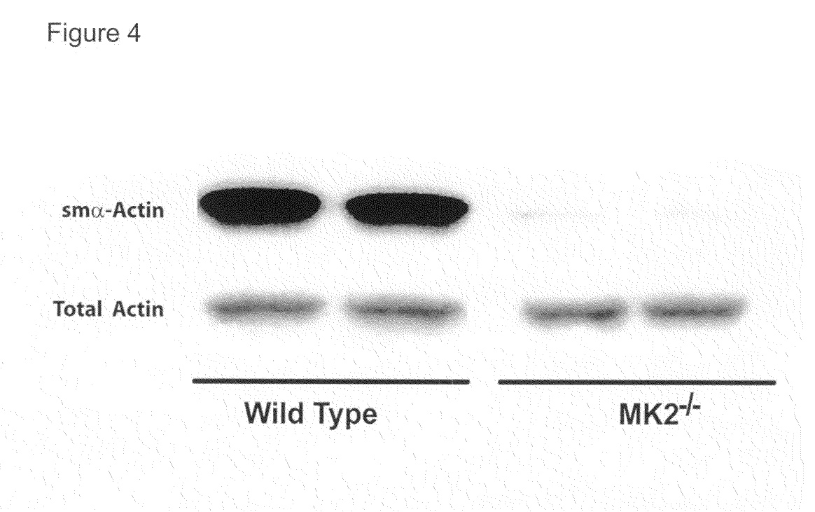

When cell lysates were immunoblotted for smα, the protein was barely detectable in MK2^{-/-} MEF, compared to WT MEF which expressed much higher levels of smα (FIG. 4). On the other hand, levels of pan-actin, a non-muscle specific form of actin, were similar in the two types of MEF (FIG. 4). These results indicate that MK2^{-/-} MEF are less differentiated into myofibroblasts than WT MEF.

### Example 8

### TGFβ Induces smα Expression in WT MEF but not in MK2^{-/-} MEF

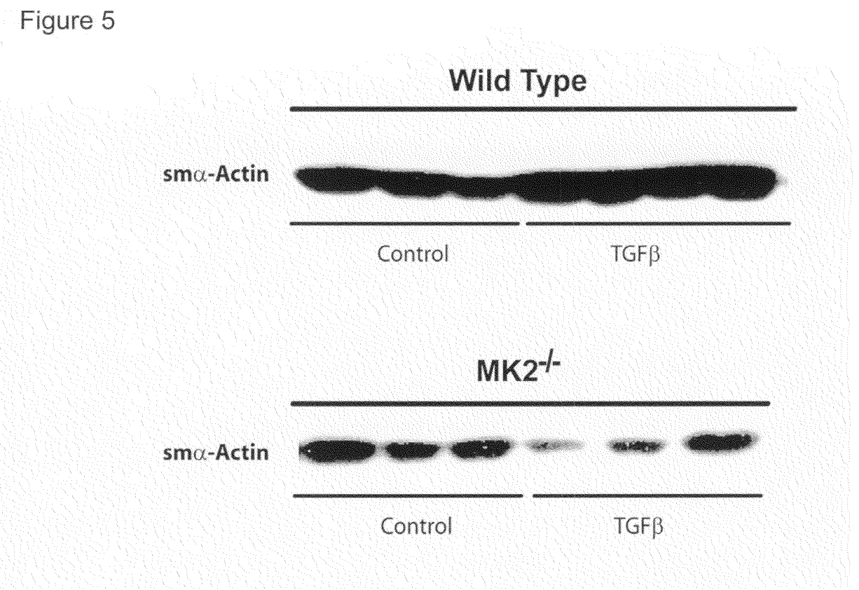

TGFβhas been implicated in the fibrotic response because of its ability to induce differentiation of fibroblasts into myofibroblasts. Whether TGFβ can induce myofibroblast differentiation in both WT MEF and MK2^{-/-} MEF was tested. Cells were growth arrested in serum free media for 24 hr then exposed to TGFβ (1 ng/mL). As shown in FIG. 5, TGFβ treatment causes WT MEF to produce more sma, but not MK2^{-/-} MEF which produce even less smα in response to TGFβ.

### Example 9

### TGFβ Exerts Opposite Effects on the Level of smα mRNA in WT Versus MK2^{-/-} MEF

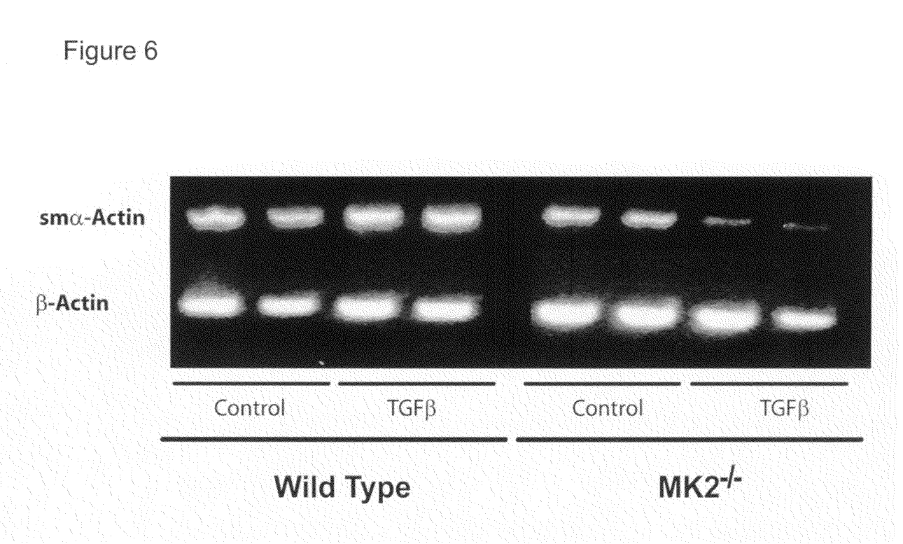

As shown in FIG. 6, the level of smα message was lower in MK2^{-/-} MEF than in WT MEF. TGFβ (1 ng/mL for 24 hr) appeared to increase the smα message level in WT MEF. However, it significantly decreased the level of smα mRNA in MK2^{-/-} MEF. This latter decrease in MK2−/− cells is consistent with the effect of TGFβ on smα protein level shown in FIG. 5.

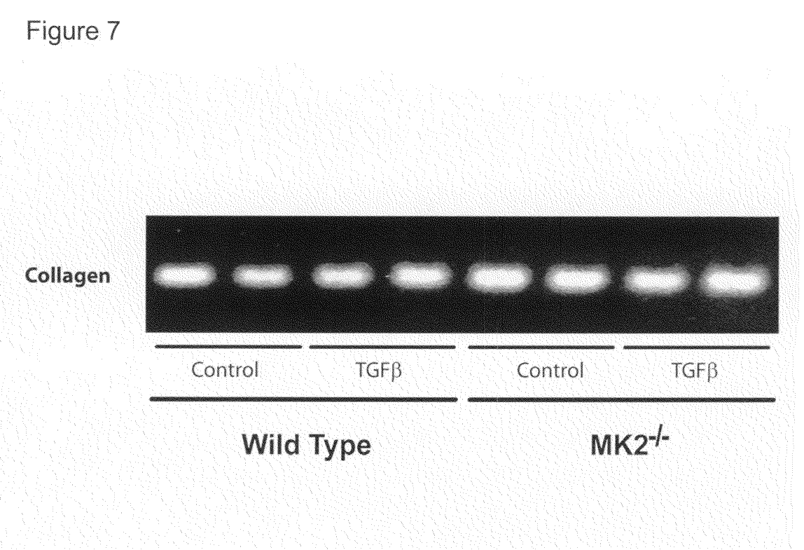

The effect of MK2 suppression on the level of smα mRNA was specific in that the levels of collagen mRNA, another key protein produced by fibroblasts, were not different between MK2^{-/-} MEF and WT MEF (FIG. 7). Furthermore, TGFβ treatment did not suppress the collagen mRNA level as it did for smα mRNA in MK2^{-/-} MEF (FIG. 7).

### Example 10

TGFβ Activates Serum Response Elements in WT MEF in a p38 Dependent Manner

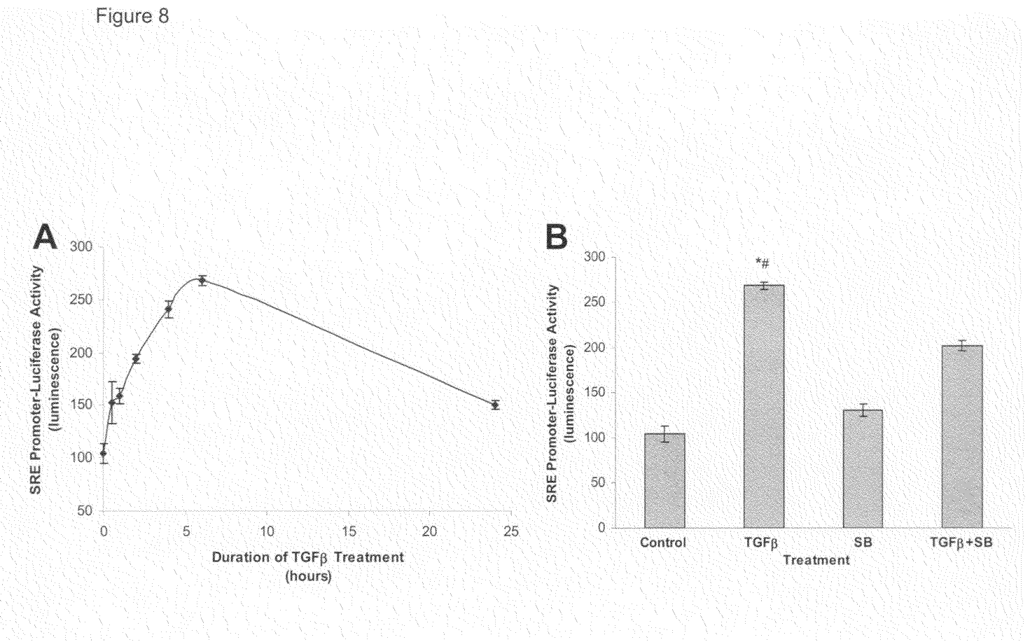

Since smα induction by several agents including TGFβ has been reported to occur through activation of serum response elements (SRE) in the smα promoter, whether this activation occurs in WT MEF was tested. As shown in FIG. 8A, TGFβ (1 ng/mL) caused activation of the SRE-luciferase, which increased with time up to 6 hours and returned to baseline by 24 hours. An inhibitor of p38 MAP kinase, SB-203580 (1 μM), partially blocked the activation of SRE-luciferase by TGFβ suggesting that p38 pathway might be involved in SRE promoter activation (FIG. 8B).

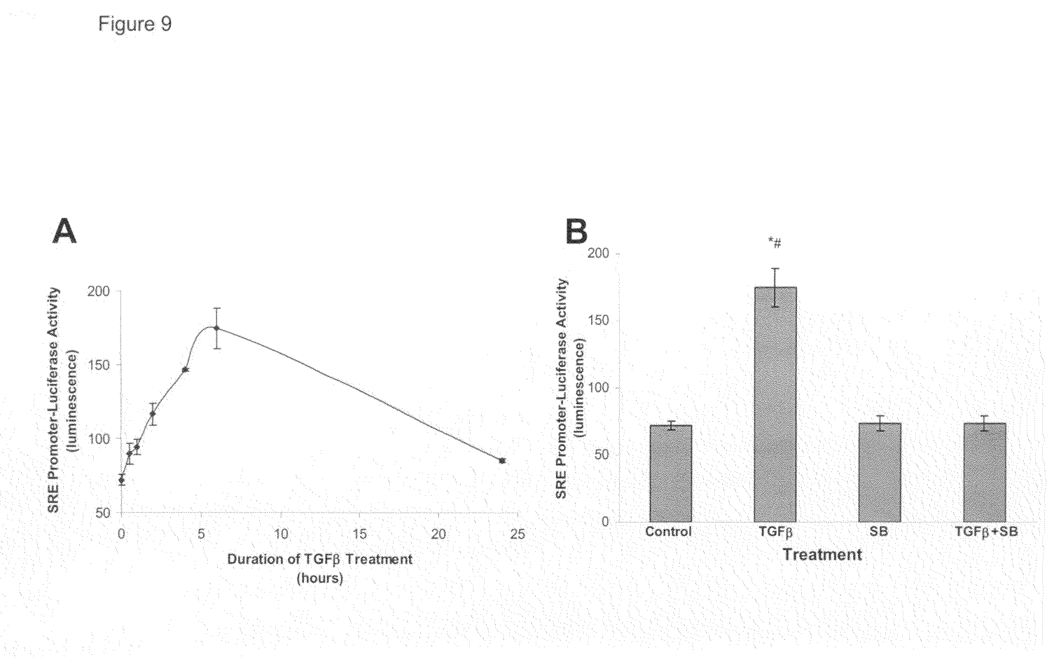

Since MK2 has been reported to phosphorylate SRF, whether the effect of MK2 suppression on smα expression is due to its effect on the SRE promoter was tested. As shown in FIG. 9A, TGFβ (1 ng/mL) caused activation of the SRE-luciferase in MK2^{-/-} MEF, increasing with time up to 6 hours and returning to baseline by 24 hours. The extent of promoter activation and its time-course were similar to those observed in WT MEF suggesting that MK2 disruption has no effect on TGFβ-induced SRE promoter activity. An inhibitor of p38 MAP kinase, SB-203580 (1 μM), completely blocked the activation of SRE-luciferase by TGFβ suggesting that p38 pathway might be involved in SRE promoter activation (FIG. 9B). The difference in inhibition level might reflect a lower level of p38 in MK2^{-/-} MEF.

### Example 11

### smα mRNA is Less Stable in MK2^{-/-} MEF than in WT MEF

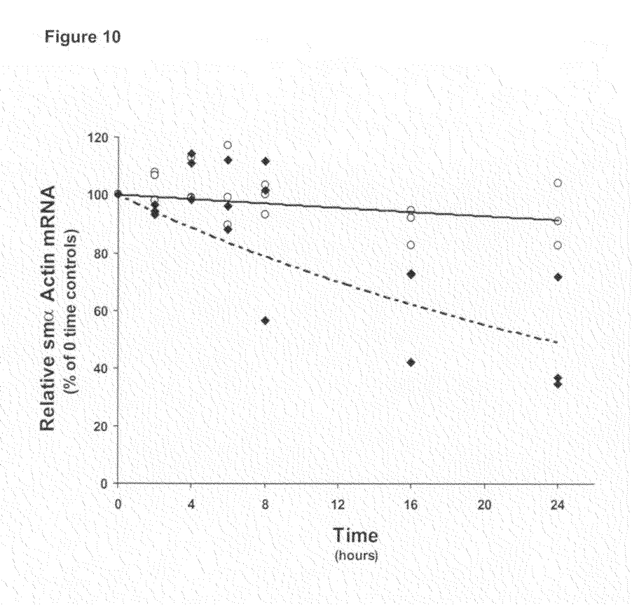

Since MK2 has been described to regulate the expression of some proteins, such as IL-6 and TNF through altering their message stability, smα mRNA was measured and normalized it to β-actin mRNA in MK2^{-/-} and WT MEF. Cells were incubated with 2.4 μM actinomycin D to inhibit transcription, and relative smα mRNA was assayed by semi quantitative RT-PCR at different time points. As shown in FIG. 10, smα mRNA was degraded at a faster (8 fold) rate in MK2^{-/-} compared to WT MEF. The half-life of smα mRNA was 23 hours in MK2^{-/-} MEF while it was 187 hours in WT MEF. These results suggest that the reason MK2^{-/-} MEF express less smα than WT MEF might be due to reduced smα message stability in these cells.

### Example 12

### Embryonic Fibroblasts from MK2^{-/-} Mice Exhibit a Higher Proliferative Rate

For proliferation assays, cells were seeded in 12-well dishes and cultured for 24, 48 and 72 h. Counting was performed after trypsinizing the cells using a Coulter counter apparatus according to manufacturer's instructions.

Since both MEF and whole animals exhibit the same characteristic inhibition of smα in response to MK2 disruption, other characteristics of fibroblasts in culture that might explain the observations made in bleomycin-treated mice were investigated. When seeded at equal density mouse embryonic fibroblasts from MK2^{-/-} mice grew at a faster rate than their wild type counterparts. This effect was observed at 10% serum and in the absence of serum, such that MK2^{-/-} were more tolerant to serum deprivation. As shown in FIG. 1 1, MK2^{-/-} MEF cell count increases faster than wild type MEF. Such increased proliferation rate might explain the excessive cellularity observed in fibrotic foci in bleomycin-treated MK2^{-/-} mouse lungs.

### Example 13

MK^{-/-} Fibroblasts are Defective in Migratory Activity

For migration assays, cells were plated in 35 mm dishes, after 24 h the monolayer was scratched with a sterile tip to make the wounding gap (time 0 h). Cells were cultured for another 24 h in 10% serum media and then fixed with 4% formaldehyde for 10 min. Four pictures were randomly taken from duplicate dishes and wound closure was compared with time 0 h to assess cell migration.

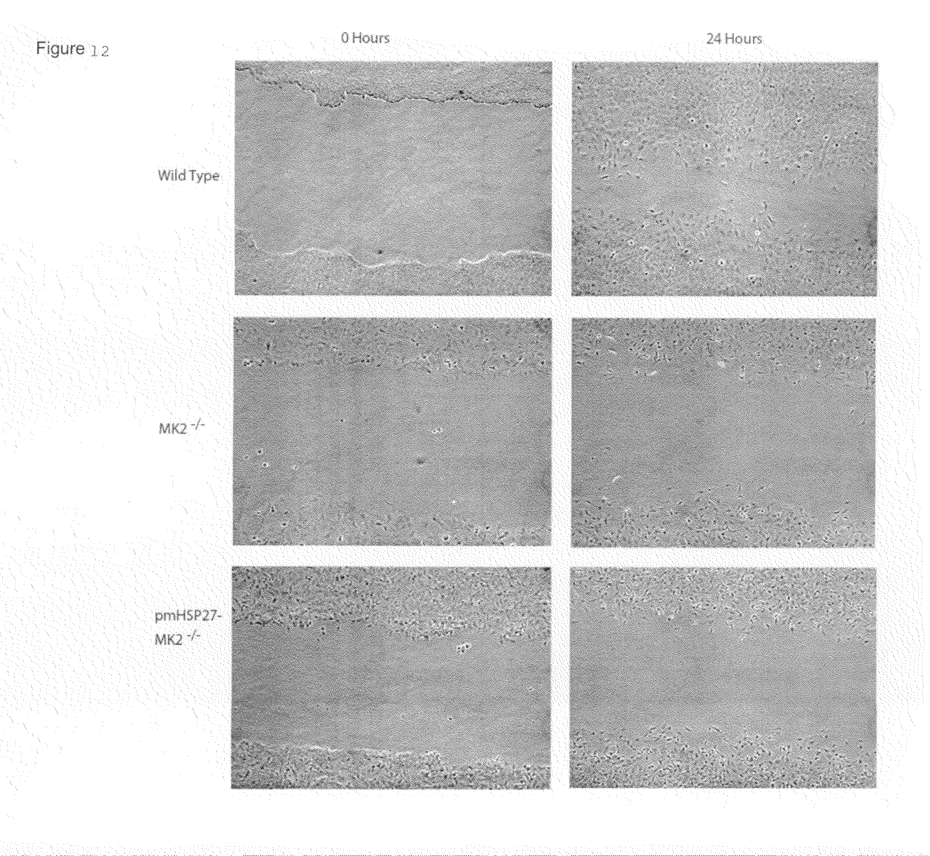

MK2^{-/-} MEF have been described to migrate more slowly than their wild type counterparts. The ability of MEF to repopulate a wound made by a sterile pipette in a tissue culture dish was assayed. As seen in FIG. 12, wild type MEF repopulated the wounded area much faster than the MK2^{-/-} cells. As MK2 is known to alter the actin cytoskeleton through its direct action on the small heat shock protein HSP27, stably transfected phosphomicmiking (pm) HSP27 MK2^{-/-} MEF were generated. It was previously demonstrated that expressing this construct stably in endothelial cells causes an increase in actin stress fibers, which is believed to represent a more contractile phenotype. While transfected MK2^{-/-} MEF expressed phosphomimicking HSP27, they were still defective in migration compared to wild type cells (FIG. 12). Furthermore, since some reports demonstrated that increased physical contractility can in turn induce the expression of smα, whatever increased contractility overexpressing pmHSP27 could cause, had no effect on the expression of α-SMA in MK2^{-/-} MEF. These results indicate that MK2 affects migration of fibroblasts, not through its effect on HSP27 phosphorylation, but possibly through a direct effect on α-SMA expression.

What is claimed is:

1. The use of a compound that increases the expression of MK2 in the manufacture of a medicament for the therapeutic treatment of a vertebrate that has been wounded.

2. The use of a compound that increases the activity of MK2 in the manufacture of a medicament for the therapeutic treatment of a vertebrate that has been wounded.

3. A method for therapeutically treating a vertebrate that has been wounded comprising administering to the vertebrate a compound that increases the expression of MK2, thereby aiding in the wound healing or wound repair processes.

4. A method for therapeutically treating a vertebrate that has been wounded comprising administering to the vertebrate a compound that increases the activity of MK2, thereby aiding in the wound healing or wound repair processes.

5. A method for screening for a compound that modulates the expression of MK2, the method comprising providing cells, contacting the cells with a test compound and assaying the cells for changes in expression of MK2 in the presence of the compound as compared to the absence of the compound.

6. A method for screening for a compound that modulates the activity of MK2, the method comprising providing cells, contacting the cells with a test compound and assaying the cells for changes in the activity of MK2 in the presence of the compound as compared to the absence of the compound.

7. A compound identified by the method according to claim 5.

8. A compound identified by the method according to claim 6.

9. A pharmaceutical composition comprising a compound according to claim 7 and a pharmaceutically acceptable carrier.

10. A pharmaceutical composition comprising a compound according to claim 8 and a pharmaceutically acceptable carrier.
